(19) United States
(12) Patent Application Publication
(10) Pub. No.: US 2010/0198181 A1
(43) Pub. Date: Aug. 5, 2010

(54) DISPOSABLE HYGIENE PRODUCTS APPLYING AN ELASTIC SPANDEX FIBER WITH HIGH POWER RETENTION

(75) Inventors: Sang-Won Cho, Seongnam-si (KR); Bo-Hyun Bang, Anyang-si (KR); Yeon-Soo Kang, Anyang-si (KR); Seung-Won Soe, Gunpo-si (KR)

Correspondence Address: NIXON & VANDERHYE, PC, 901 NORTH GLEBE ROAD, 11TH FLOOR, ARLINGTON, VA, 22203, US

(73) Assignee: HYOSUNG CORPORATION, Seoul (KR)

(21) Appl. No.: 12/671,541

(22) PCT Filed: Jun. 20, 2008
(86) PCT No.: PCT/KR2008/003502

§ 371 Date: Feb. 1, 2010

(30) Foreign Application Priority Data
Jul. 6, 2007 (KR) 10-2007-0068230
Dec. 7, 2007 (KR) 10-2007-0127139

(51) Int. Cl. A61F 13/49; C08G 18/32

(52) U.S. Cl. 604/385.25; 528/61

(57) ABSTRACT

The present invention relates to disposable hygiene products such as a diaper or a sanitary napkin applying an elastic spandex fiber with high power retention. A polyurethane-based spandex fiber is produced by reacting an organic diisocyanate with a polymer diol to prepare a prepolymer, dissolving the prepolymer in an organic solvent, and reacting the resulting solution with a diamine and a monoamine. A disposable hygiene product having better functionality and wearing comfort is provided by using a spandex fiber as an elastic yarn, wherein the spandex fiber has power retention of 70% or more.

### TECHNICAL FIELD

The present invention relates to disposable hygiene products using spandex fibers with higher power retention than the conventional fibers, thereby improving wearing comfort and functionality.

### BACKGROUND ART

Disposable hygiene products are increasingly used because of their convenience. A disposable diaper, for example, varies from baby diaper to adult diaper in its size, usability and shape. Recently, a pull-up type in a form of short pants is more widely used than a simple straight diaper. An elastic body is used for this type of disposable diaper for elasticity and wearing comfort. A rubber-based elastic yarn or a polyurethane-based spandex fiber is widely used, mainly on the flap part of the legs and the cuff part, and stretchable nonwoven fabric is used on the side panels and on the waist band. The flap part of the legs and the cuff part have impact on the functionality and wearing comfort of diapers, depending on the physical properties of elastic yarns used. And diaper manufacturers are now making ceaseless efforts to develop an elastic yarn having improved flexibility as well as wearing comfort in order to produce a higher value-added diaper.

The cuff part in disposable diapers primarily prevents the liquid discharge from leaking out and the flap part of the legs secondly prevents leakage of the liquid discharge. These two parts have direct influence on wearing comfort as they are closely attached to the body. If the elastic yarn used is too tight, it leads to deterioration in wearing comfort, and causing red mark around the legs, whereas if too loose, the liquid discharge would leak out of the diaper. That is, if the elastic yarn too much extended is applied to the diaper in order to prevent the liquid discharge from leaking outside, it can increase the tightness of the elastic yarn, but lead to uncomfortable wearing, and causing red mark and pain on the skin. On the other hand, if an elastic yarn relatively less extended is applied to the diaper, the wearing comfort would be improved, but causing a problem of leaking. Therefore, in order to improve both the functionality and wearing comfort, an elastic yarn is required to be flexible enough to extend easily when being pulled, and to be closely attached to the body all the time. This relates closely to power retention of an elastic yarn.

The power retention in this description refers to a ratio value of S2 to S1 (S2/S1×100%). S1 is a value of tension at 200% extension (for example, when 10 cm is extended to 30 cm) at the fifth time when an elastic yarn is repeatedly extended to 300% (for example, 10 cm is extended to 40 cm.) five times. And S2 is a value of tension at 200% extension (for example, when 10 cm is extended to 30 cm.) when the elastic yarn is shrunk. The power retention (S2/S1×100%) shows how easily an elastic yarn is extended and how closely it is attached to the skin. The bigger the value is, with less power an elastic yarn is extended smoothly and when shrunk, it is closely attached to the skin all the time, achieving both the functionality and wearing comfort.

Among the conventional elastic yarns, a rubber-based elastic yarn has the best functionality and wearing comfort. A rubber-based elastic yarn has high power retention of 70% to 80% so that it has better wearing comfort and functionality than any other elastic yarns used before. It has drawbacks, however, in that the process quality is relatively inferior to that of a spandex fiber due to a yarn cutting during the production of diapers, and has a poor tolerance to chemicals. In terms of economic feasibility, the price per unit weight of rubber-based elastic yarn is lower compared to a spandex fiber, but in effect, the amount of rubber-based elastic yarn used to make one diaper is more than three times that of a spandex fiber, so in practice when making one diaper, using a rubber-based elastic yarn is less economical than using a spandex fiber.

Meanwhile, most of the spandex fibers used in disposable hygiene products such as diapers or sanitary napkins are unable to achieve both the wearing comfort and functionality at the same time, since they have power retention of less than 65%. That is, in case that an elastic yarn is applied loosely to improve wearing comfort, the possibility of leakage of liquid discharge increases, while an elastic yarn is applied tightly in order not to cause leakage, the tightness is such great that it leads to red mark on the skin.

Therefore, if using a spandex fiber with high power retention which is comparable to that of a rubber-based elastic yarn in disposable hygiene products such as diapers or sanitary napkins, disposable hygiene products with better qualities may be produced, thereby solving all the drawbacks mentioned above.

Generally, a spandex fiber is widely known as a polyurethane fiber with high elasticity prepared from polymer in which 85 wt % or more of polyurethane resin having segment is included. The conventional spandex fiber used in disposable hygiene products such as diapers and sanitary napkins is a general-purpose spandex fiber produced by reacting an organic diisocyanate with a polymer diol to prepare a prepolymer, dissolving the prepolymer in an organic solvent, and reacting the resulting solution with a diamine and/or a monoamine. It is used diphenylmethane-4,4′-diisocyanate, diphenylmethane-2,4-diisocyanate, toluene diisocyanate, 1,6-diisocyanatohexane, 2,2-bis(4-isocyanatophenyl)propane, 1,1′-methylenebis(4-isocyanatocyclohexane), 1,4-diisocyanatocyclohexane or a mixture of them as a diisocyanate. 100% of poly(tetramethylene ether)glycol is usually used as a polymer diol to produce a spandex with power retention of less than 65%.

In case of the above mentioned general-purpose spandex fiber, however, it is not able to achieve both the functionality and wearing comfort at the same time. Hence a skill that enables a spandex fiber to have high power retention similar to that of a rubber-based elastic yarn is highly important. In the prior art, a poly(tetramethylene ether)glycol copolymer using a neopentyl glycol or a 3-methyltetrahydrofuran is used instead of 100% poly(tetramethylene ether)glycol as a polymer diol, thereby increasing power retention. For example, U.S. Pat. No. 5,000,899 discloses a method of producing spandex. In the method, after using a random copolymer of a 3-methyltetrahydrofuran and a tetrahydrofuran to form soft segments, ethylenediamine and 2-methylpentamethylenediamine are used as chain extenders to produce a spandex fiber. However, in case that spandex is produced using the method, the problem of adhesion during the storage of the produced yarn may occur due to the high adhesion between the yarns, and if applied to the diaper manufacturing process, it may lead to recurrent yarn breakage because of the poor quality of yarn unwinding, thereby making the process operation hard.

Another related art, U.S. Patent No. 2006/281891A, discloses a method of producing spandex by using a poly(tetramethylene ether)glycol as an initiator and a polymer diol produced by polymerizing a mixture of alkylene oxide and lactone monomer. By using the method, a spandex may have high power retention and good quality of a yarn unwinding as adhesion between the yarns is relatively low.

Therefore, applying the spandex produced on the basis of the above-mentioned skill of producing spandex with high power retention to disposable hygiene products such as diapers and sanitary napkins achieves both the functionality and wearing comfort, and much more economical products may be produced than that made by using a rubber-based elastic yarn.

### DISCLOSURE OF INVENTION

### Technical Problem

The object of the present invention is to provide a spandex having power retention of 70% or more, and to provide disposable hygiene products applying a spandex having power retention comparable to that of a rubber-based elastic yarn in order to achieve both the functionality and wearing comfort which are required for disposable hygiene products.

### Technical Solution

A spandex elastic yarn according to the present invention is applicable to disposable hygiene products such as a diaper and a sanitary napkin, wherein the spandex fiber has power retention of 70% or more, thereby having better functionality and wearing comfort than the conventional one.

According to the preferred embodiment of the present invention, disposable hygiene products using a polyurethane-based spandex elastic yarn with power retention of 70% or more may be provided.

According to another preferred embodiment of the present invention, a diaper applying a polyurethane-based spandex elastic yarn with power retention of 70% or more is provided, and particularly the polyurethane-based spandex elastic yarn may be applied to the flap part of the legs and the cuff part of the diaper. Further, in addition to the flap part of the legs and the cuff part of the diaper, the elastic yarn may be applied properly to the other parts of the diaper, that is, to the waist part or the body part of a diaper without any specific limitations.

According to another preferred embodiment of the present invention, the elastic yarn is a polyurethane-based elastic yarn produced by reacting an organic diisocyanate with a polymer diol to prepare a prepolymer, dissolving the prepolymer in an organic solvent, and reacting the resulting solution with a diamine and a monoamine.

According to another embodiment of the present invention, the polymer diol is a polyetherester diol that polymerizes a mixture of alkylene oxide and lactone monomer under a poly(tetramethylene ether)glycol as an initiator.

According to another embodiment of the present invention, the polymer diol is a copolymer that polymerizes a mixture of an alkylene oxide and a lactone monomer under a poly(tetramethylene ether)glycol as an initiator, or a poly(tetramethylene ether)glycol is added to the copolymer at a ratio of 0.1 to 50 wt % of the total weight of the polymer diol.

According to the present invention, the said organic diisocyanate is selected among a group consisting of diphenylmethane-4,4′-diisocyanate, diphenylmethane-2,4-diisocyanate, toluene diisocyanate, 1,6-diisocyanatohexane, 2,2-bis(4-isocyanatophenyl)propane, 1,1′-methylenebis(4-isocyanatocyclohexane), 1,4-diisocyanatocyclohexane and from a mixture of them.

According to the present invention, a polyurethane-based spandex elastic yarn having power retention of 70% or more may be applied to a sanitary napkin.

### ADVANTAGEOUS EFFECTS

If a spandex yarn with high power retention of the present invention is applied to disposable hygiene products such as a diaper or sanitary napkins, products may be produced with functionality and wearing comfort, in which the problem of red mark occurred when using the conventional spandex fiber may be solved, and the liquid discharge may be held inside the pad of a diaper and at the same time making a user feel fine and comfortable. And hopefully, replacing a relatively pricey rubber-based elastic yarn with spandex yarn may contribute to economic efficiency improvement in the diaper manufacturing industry.

### BRIEF DESCRIPTION OF THE DRAWINGS

FIG. 1 shows schematically a part plane FIGURE of a disposable diaper applying an elastic yarn of the present invention.

### BEST MODE FOR CARRYING OUT THE INVENTION

Hereinafter, the present invention will be described in detail.

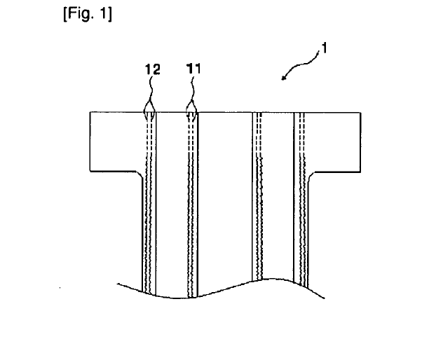

FIG. 1 shows a schematic embodiment of a disposable diaper applying a spandex elastic yarn of the present invention to the flap part of the legs 12 and to the cuff part 11. The cuff part in the diaper primarily prevents the liquid discharge of the diaper from leaking out thereof, and the flap part of the legs secondly prevents the leakage of liquid discharge. Those two parts, the flap part and cuff part, have direct influence on the wearing comfort as they are closely attached to the skin. If an elastic yarn used is too tight, the wearing comfort deteriorates and causing a red mark around the legs, while if too loose, the liquid discharge may leak out of diapers. Therefore, in order to achieve both the wearing comfort and functionality, an elastic yarn is required to have the physical properties that are flexible enough to extend easily when being pulled, and that at the same time are closely attached to the skin all the time. The properties relates closely to power retention of an elastic yarn. Thus it is desirable for the spandex elastic yarn to have power retention of 70% or more similar to that of a rubber-based elastic yarn.

The spandex fiber may be produced by reacting an organic diisocyanate with a polymer diol to prepare a prepolymer, dissolving the prepolymer in an organic solvent, and reacting the resulting solution with a diamine and a monoamine as disclosed in the prior art.

A polymer diol constituting a soft portion of the polyurethane may use a polymer diol produced by polymerizing a mixture of alkylene oxide and lactone monomer under a poly(tetramethylene ether)glycol as an initiator. Alternatively a poly(tetramethylene ether)glycol copolymer containing a 3-methyltetrahydrofuran or a neopentyl glycol may be used.

In the present invention, it is desirable that alkylene oxide be selected among a group of ethylene oxide, 1,2-propylene oxide and 1,2-butylene oxide. Lactone monomer may be butylolactone or caprolactone, preferably caprolactone. It is preferable that the total content of alkylene oxide and lactone monomer be 20 to 80 wt % in the molecule of polymer diol. In case the total content is less than 20 wt %, power retention of a spandex deteriorates, and in case more than 80 wt %, problems such as yarn cutting occur during the production of yarns. It is preferable that the molar ratio of an alkylene oxide to a lactone monomer be 30/70 to 95/5. If the ratio is less than 30/70, power retention becomes poor, and if more than 95/5, the strength of yarns weakens, causing yarn cutting.

It is preferable that a number average molecular weight of polymer diol in the present invention be 800 to 4000. If it is less than 800, elongation at break decreases, whereas if the molecular weight exceeds 4000, viscosity increases, and synthesizing a urethane resin becomes difficult. Organic diisocyanate may preferably be selected among a group of diphenylmethane-4,4′-diisocyanate, diphenylmethane-2,4′-diisocyanate, toluene diisocyanate, 1,6-diisocyanatohexane, 2,2-bis(4-isocyanatophenyl)-propane, 1,1′-methylenebis(4-isocyanatocyclohexane), 1,4-diisocyanatocyclohexane and from a mixture of them.

Diamines used as a chain extender may comprise ethylenediamine, propylenediamine, hydrazine, and so on. Monoamine used as a chain terminator may comprise diethylamine, monoethanolamine, dimethylamine, and so on.

The spandex elastic yarn prepared in the present invention is 400 to 1800 deniers under ordinary manufacturing conditions and is applied to disposable hygiene products. The organic diisocyanate reacts with a polymer diol at a NCO/OH molar ratio of 1.3 to 2.3 to obtain a polyurethane prepolymer. The resulting prepolymer is dissolved in an organic solvent such as dimethylacetamide and dimethylformamide. And then the diamine such as ethylenediamine as a chain extender and monoamine such as diethylamine as a chain terminator is added at a NCO/(NH₂+NH) equivalent ratio of 0.90 to 1.15 to prepare the polyurethane resin solution. A spandex fiber is produced by dry spinning the resulting solution with hot wind at 150° C. to 300° C. in a dry spinning cell.

Hereinafter, the present invention will be described in detail with reference to drawings and embodiments. These embodiments are provided only for the illustrative purpose, and it should not be construed that the scope of the invention is limited thereto.

The effects of the invention may be proved by applying the spandex elastic yarn having power retention of 70% or more to a diaper as a representative example of disposable hygiene products and by evaluating it as in the following examples below. The experiment to test an elastic yarn applied to a disposable diaper was conducted with six kinds of elastic yarns, as in the table below.

TABLE 1
Regarding to Table 1: An elastic yarn used
in the production of a disposable diaper.

| Experiment | Kinds of elastic yarns |
|---|---|
| Example 1 | A spandex elastic yarn produced by using only |
| | a polyetherester diol as a polymer diol that |
| | polymerizes a mixture of an alkylene oxide and |
| | a lactone monomer using a poly(tetramethylene |
| | ether)glycol as an initiator |
| Example 2 | A spandex elastic yarn produced by using a |
| | poly(tetramethylene ether(glycol(molecular |
| | weight 1800) at 20 wt % of the total weight of |
| | polymer diol, and a polyetherester diol at 80 |
| | wt % of the total weight of polymer diol that |
| | polymerizes a mixture of an alkylene oxide and |
| | a lactone monomer using a poly(tetramethylene |
| | ether)glycol as an initiator |
| Example 3 | A spandex elastic yarn produced by using a |
| | copolymer of a 3-methyltetrahydrofuran and a |
| | tetrahydrofuran(13 mol % of 3- |
| | methyltetrahydrofuran, 87 mol % of |
| | tetrahydrofuran, molecular weight 3000) |
| Comparative | A spandex elastic yarn produced by using 100% |
| example 1 | of poly(tetramethylene ether)glycol as a |
| | polymer diol, which is generally used in the |
| | diapers. |
| Comparative | An other kind of spandex elastic yarn produced |
| example 2 | by using 100% of poly(tetramethylene |
| | ether)glycol, which is generally used in the |
| | diapers. |
| Comparative | A rubber-based elastic yarn(45G) |
| example 3 | |

A disposable diaper is prepared by using the above six kinds of elastic yarns. An elastic yarn is 700 deniers and a rubber-based elastic yarn is 45 G. In this case, a spandex fiber and a rubber-based elastic yarn are extended to 200% (DR=3) and 150% (DR=2.5), respectively, and two strands are applied to the flap part of the legs and the cuff part, respectively. Power retention of the six kinds of diapers produced is evaluated depending on different extensions. And in practice, 100 babies are chosen to be tested about how often the red mark occurs when wearing each diaper, and the results were evaluated with bare eyes. Preferences on the six kinds of diapers are valued depending on the customer satisfaction measurement relating to wearing comfort and functionality.

Evaluation of Power Retention of a Spandex Fiber

The gauge length was set to 2 inches using INSTRON 5565 manufactured by Instron Corp., and extension and contraction of five elastic yarn long fiber samples was repeated at a constant drawing rate of 500 mm/min five times so that the samples were extended to 0 to 300%. The ratio of stress (S2) when the contraction was conducted to stress (S1) when the sample was extended 200% while the fifth extension was performed was turned into percentage to obtain power retention.

Power retention(%)=S2/S1×100 Equation 1:

Evaluation of Power Retention of Diapers

The evaluation of power retention depending on different extensions in each state of products was also conducted using INSTRON 5565. First of all, the first gauge length was set to 50% of the length of a diaper being fully unfolded. And the flap part of the legs is cut carefully into 20 mm-wide rectangles and then the rectangles are fixed on the upper and lower part of INSTRON, and they are extended to 95% at a constant drawing rate of 500 mm/min. Power retention is measured at the extension rate of 80%, 90% and 95%. Basically, if power retention at each extension gets bigger, the tightness when wearing a diaper increases. Gentle increase of power retention in proportion with the extension rise of 80%→90%→95% means a smooth elongation, and making a user feel fine and comfortable when moving while wearing a diaper.

Evaluation of the Degrees of Red Mark

In order to evaluate red mark occurrence, 100 toddlers under age 3 were chosen at random without considering condition such as a body shape. Those kids wore the diapers for three days and were surveyed in a written form if red mark appeared on the skin. Appraisal standards are shown in table 2 below.

TABLE 2

| | Power | Tension in a diaper state (g) | | | | |
|---|---|---|---|---|---|---|
| | retention | | | The increase | | |
| | in fiber | | | in tension | The | |
| Test | state | Extension | Tension | to 80% | degrees of | |
| items | (%) | rate (%) | (g) | extension (%) | red mark | Preference |
| Ex. 1 | 82 | 80 | 84.5 | — | 1.5 | 868 |
| | | 90 | 92.7 | 8.8 | | |
| | | 95 | 105.3 | 19.8 | | |
| Ex. 2 | 78 | 80 | 84.3 | — | 8.0 | 350 |
| | | 90 | 93.8 | 10.9 | | |
| | | 95 | 107.7 | 22.3 | | |
| Ex. 3 | 80 | 80 | 87.9 | — | 3.5 | 678 |
| | | 90 | 106.4 | 10.1 | | |
| | | 95 | 137.8 | 21.7 | | |
| Comp. | 59 | 80 | 87.9 | — | 70.0 | 122 |
| Ex. 1 | | 90 | 106.4 | 17.4 | | |
| | | 95 | 137.8 | 36.2 | | |
| Comp. | 60 | 80 | 84.3 | — | 62.5 | 128 |
| Ex. 2 | | 90 | 103.4 | 18.4 | | |
| | | 95 | 133.7 | 36.9 | | |
| Comp. | 88 | 80 | 85.3 | — | 1.0 | 854 |
| Ex. 3 | | 90 | 92.4 | 8.8 | | |
| | | 95 | 100.4 | 18.0 | | |

Regarding to Table 2: Appraisal standards for red mark occurrence
(Question) How badly the red mark appeared on the skin after wearing diapers for three days?

Evaluation of Product Preferences

The evaluation of product preferences on the basis of wearing comfort and functionality was conducted at the same time with the evaluation of the degrees of red mark. The product preferences were evaluated by making toddlers wear six kinds of diapers for three days and were asked to make assessment on each diaper sample on the 20^{th} day after the test started. The preference rankings are shown in Table 3 below.

TABLE 3

Preferences (ranking)

| | 1 | 2 | 3 | 4 | 5 | 6 |
|---|---|---|---|---|---|---|
| Individual | 10 | 8 | 6 | 4 | 2 | 0 |
| evaluation (unit: point) | | | | | | |

| | |
|---|---|
| 100 individuals' total | The sum total of 100 individuals' |
| evaluation (unit: point) | evaluation (the highest point: 1000) |

Regarding to Table 3: Evaluation of product preferences on diapers

### Example 1

The spandex fiber was obtained by using only polyetherester diol as a polymer diol that polymerizes a mixture of alkylene oxide and lactone monomer using a poly(tetramethylene ether)glycol as an initiator. Power retention of the spandex fiber was evaluated and diapers were produced using the same elastic yarn. Power retention of the diapers was evaluated depending on different extensions, and the degrees of red mark and product preferences were assessed with the 100 toddlers selected to wear the diapers. The results of measurements are shown in table 4.

### Example 2

The procedure of example 1 was repeated, except using a spandex elastic yarn produced by using a mixture of a polyetherester diol and a poly(tetramethylene ether)glycol (molecular weight of 1800) at the mass ratio of 80:20, which the polyetherester diol is polymerized by using a mixture of an alkylene oxide and a lactone monomer under a poly(tetramethylene ether)glycol as an initiator.

### Example 3

The procedure of Example 1 was repeated except using a spandex elastic yarn produced by using a copolymer of a 3-methyltetrahydrofuran and a tetrahydrofuran (13 mol % of 3-methyltetrahydrofuran, 87 mol % of tetrahydrofuran, molecular weight of 3000).

### Comparative Example 1

The procedure of Example 1 was repeated except using a general-purpose spandex fiber (using 100% poly(tetramethylene ether)glycol as a polymer diol) generally applied to the production of diapers.

### Comparative Example 2

The procedure of Example 1 was repeated except using another kind of general-purpose spandex fiber (using 100% poly(tetramethylene ether)glycol as a polymer) generally applied to the production of diapers.

### Comparative Example 3

As an elastic yarn, a rubber-based elastic yarn (45 G) which is widely used to produce diapers was used. And the procedure of Example 1 was repeated, except as mentioned above extending the rubber-based elastic yarn to 150% to be applied to diapers.

TABLE 4
Regarding to Table 4: The evaluation results of
diapers applying the six kinds of elastic yarns

| | Individual evaluation | Total evaluation of 100 |
|---|---|---|
| | (unit: point) | individuals(unit: point) |
| Nothing | 0 | The sum total of 100 |
| | | individuals' evaluation |
| | | \textcircled{1} 0~10: excellent |
| | | \textcircled{2} 10~40: good |
| | | \textcircled{3} Over 40: inferior |
| A little | 0.5 | |
| Terribly | 1 | |

### INDUSTRIAL APPLICABILITY

A spandex yarn with high power retention of the present invention is applied to disposable hygiene products such as a diaper or sanitary napkins. The products may be produced with functionality and wearing comfort, in which the problem of red mark occurred when using the conventional spandex fiber may be solved, and the liquid discharge may be held inside the pad of a diaper and at the same time making a user feel fine and comfortable. And hopefully, replacing a relatively pricey rubber-based elastic yarn with spandex yarn may contribute to economic efficiency improvement in the diaper manufacturing industry.

1. Disposable hygiene products using a polyurethane-based spandex elastic yarn with power retention of 70% or more.

2. Disposable hygiene products according to claim 1, wherein the disposable hygiene product is a diaper, and the elastic yarn is applied to the flap part of the legs and/or the cuff part.

3. Disposable hygiene products according to claim 1, wherein the elastic yarn is a polyurethane-based spandex fiber produced by reacting an organic diisocyanate with a polymer diol to prepare a prepolymer, dissolving the prepolymer in an organic solvent, and reacting the resulting solution with a diamine and a monoamine.

4. Disposable hygiene products according to claim 3, wherein the polymer diol is a polyetherester diol that polymerizes a mixture of an alkylene oxide and a lactone monomer using a poly(tetramethylene ether)glycol as an initiator.

5. Disposable hygiene products according to claim 4, wherein the polymer diol is a copolymer that polymerizes a mixture of an alkylene oxide and a lactone monomer under a poly(tetramethylene ether)glycol as an initiator, or a poly(tetramethylene ether)glycol is added to the copolymer at the ratio of 0.1 to 50 wt % of the total weight of the polymer diol.

6. Disposable hygiene products according to claim 4, the organic diisocyanate is selected among a group consisting of diphenylmethane 4,4-diisocyanate, diphenylmethane-2,4-diisocyanate, toluene diisocyanate, 1,6-diisocyanatohexane, 2,2-bis(4-isocyanatophenyl)propane, 1,1-methylenebis(4-isocyanatocyclohexane), 1,4-diisocyanatocyclohexane and from a mixture of them.
